(19) United States
(12) Patent Application Publication
(10) Pub. No.: US 2006/0259153 A1
(43) Pub. Date: Nov. 16, 2006

(54) Artificial knee with cushion function

(75) Inventors: Chuen-Sen Harn, Xin-Zhuan City (TW); Rueng-Jie Zhen, Xin-Zhuang City (TW)

Correspondence Address: MORRIS MANNING MARTIN LLP, 3343 PEACHTREE ROAD, NE, 1600 ATLANTA FINANCIAL CENTER, ATLANTA, GA, 30326, US

(73) Assignee: Chang-Yu Mechanical System Ltd., Xin-Zhuang City (TW)

(21) Appl. No.: 11/128,850

(22) Filed: May 13, 2005

(51) Int. Cl. A61F 2/64; A61F 2/74

(52) U.S. Cl. 623/44; 623/35

(57) ABSTRACT

An artificial knee has a supporting portion, a knee portion, and a leg portion. The knee portion has a saddle-like body, a front knee pivotally connected to the saddle-like body and a rear knee. The leg portion has a pneumatic cylinder received in the receiving space in the leg portion. Pivotal movement of the saddle-like body relative to the front knee is able to activate the pneumatic cylinder to provide a cushion effect to a load on top of the supporting portion.

### BACKGROUND OF THE INVENTION

1. Field of the Invention

The present invention relates to an artificial knee, and more particularly to an artificial knee with cushion function so that the user of the artificial knee is able to comfortably wear the artificial knee.

2. Description of Related Art

Artificial knee is aimed to provide the amputee a function with which the user is able to comfortably walk with an artificial leg. That is the currently available artificial knee can only imitate a bending or rotation function of a natural knee without the consideration of bearing the body weight within a certain rotation angle of the artificial knee. Therefore, the amputee wearing the artificial knee is not able to walk comfortably due to a lack of cushion function. Although there are patents disclosing artificial knees with cushion function to provide the user better conditions to wear the artificial knee, the cushion mechanism inside these artificial knees is not adjustable to adapt to different situations the user is expected to confront. Therefore, the current artificial knee needs to be improved.

Other objects, advantages, and novel features of the invention will become more apparent from the following detailed description when taken in conjunction with the accompanying drawings.

### SUMMARY OF THE INVENTION

The primary objective of the present invention is to provide an improved artificial knee that is able to allow the user to bend and provide safety to the user when the body weight of the user is on top of the artificial knee within a predetermined angle.

Another objective of the present invention is to provide a pneumatic cylinder inside the artificial knee. The cushion of the pneumatic cylinder is able to provide a damping effect to the user and is adjustable so that the user is able to change the damping effect to adapt to different situations.

Another objective of the present invention is to provide a cover on a side of the artificial knee such that a maintenance access is provided for replacement/maintenance of the elements inside the artificial knee.

In order to accomplish the objective, the artificial knee of the present invention includes a supporting portion for connection to the thigh of the user, a knee portion sandwiched between the supporting portion and a leg portion to form a four link-rod mechanism, a hollow leg portion to receive therein a cylinder which is connected to the knee portion via a supporting rod and a connection portion formed on a bottom of the leg portion for connection to an extension of the user's limb, wherein the cylinder is provided with a push rod pivotally connected to the supporting rod so as to adjust the cushion of the cylinder.

Still, another objective of the present invention is that the cylinder also has multiple adjusting portions received in the cover to adjust airflow flux flowing between a top chamber and a bottom chamber inside the cylinder such that the user is able to adjust the cushion of the cylinder.

Examples of the present invention will be given for a better understanding of the present invention with reference to the drawings, in which:

### BRIEF DESCRIPTION OF THE DRAWINGS

FIG. 1 is a side view of the artificial knee of the present invention;

FIG. 2 is a schematically cross-sectional view of the artificial knee of the present invention;

FIG. 2A is an exploded perspective view of a portion of the artificial knee in FIG. 2;

FIG. 3 is a side view of the artificial knee of the present invention with a portion thereof exploded;

FIG. 4 is a schematic side plan view showing the initial status of the knee portion;

FIG. 5 is a schematic side plan view showing the operational status of the knee portion;

FIG. 6 is a schematic side plan view showing still a different operational status of the knee portion;

FIG. 7 is a schematic cross-sectional view of a different embodiment of the artificial knee of the present invention;

FIG. 8 is a schematic cross-sectional view of still a different embodiment of the artificial knee of the present invention;

FIG. 9 is a schematic cross-sectional view of still a different embodiment of the artificial knee of the present invention; and

FIG. 10 is a schematic cross-sectional view of still a different embodiment of the artificial knee of the present invention.

### DETAILED DESCRIPTION OF PREFERRED EMBODIMENT

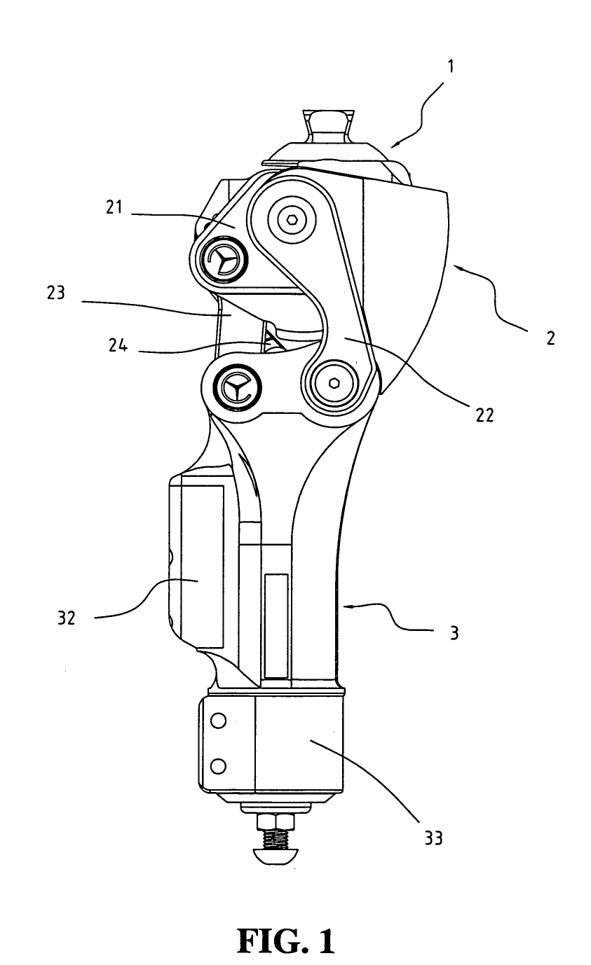

With reference to FIG. 1, it is noted that the artificial knee in accordance with the present invention includes a supporting portion 1, a knee portion 2, a leg portion 3, and a pneumatic cylinder 4.

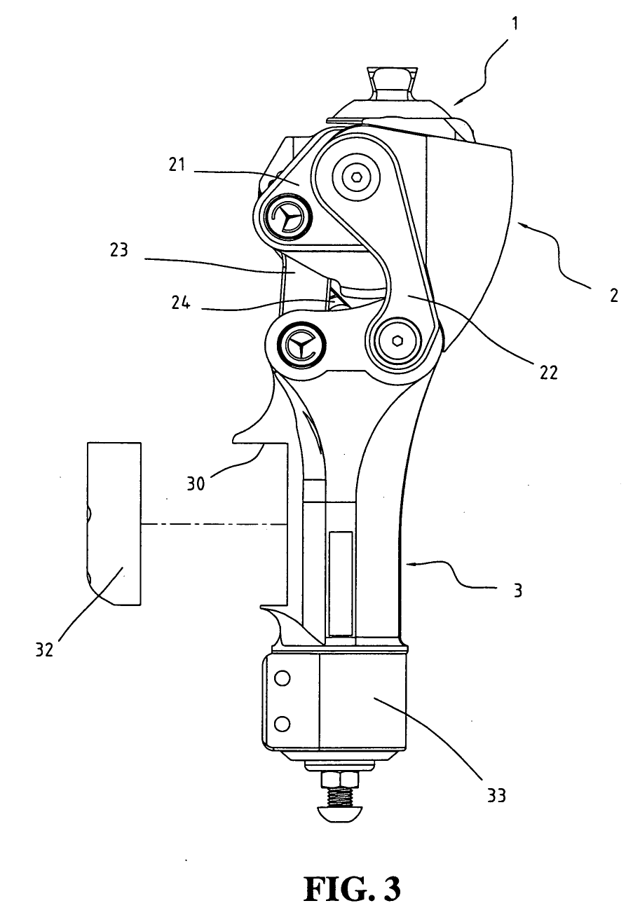

With reference to FIGS. 1, 2, 2A and 3, it is noted that the supporting portion 1 is mounted on top of the knee portion 2 for connection to a user's thigh. The knee portion 2 is provided with a saddle-like body 21, a front knee 22, and a rear knee 23 opposite to the front knee 22. The saddle-like body 21 is pivotally and respectively connected to a respective top of the front knee 22 and the rear knee 23. A respective bottom of the front knee 22 and the rear knee 23 is pivotally connected to the leg portion 3 so as to form a four-link-rod mechanism imitating the human knee operational track. A supporting rod 24 is pivotally connected to the supporting portion 1. However, in different embodiment, the supporting rod 24 may be pivotally connected to either the front knee 22 or the rear knee 23.

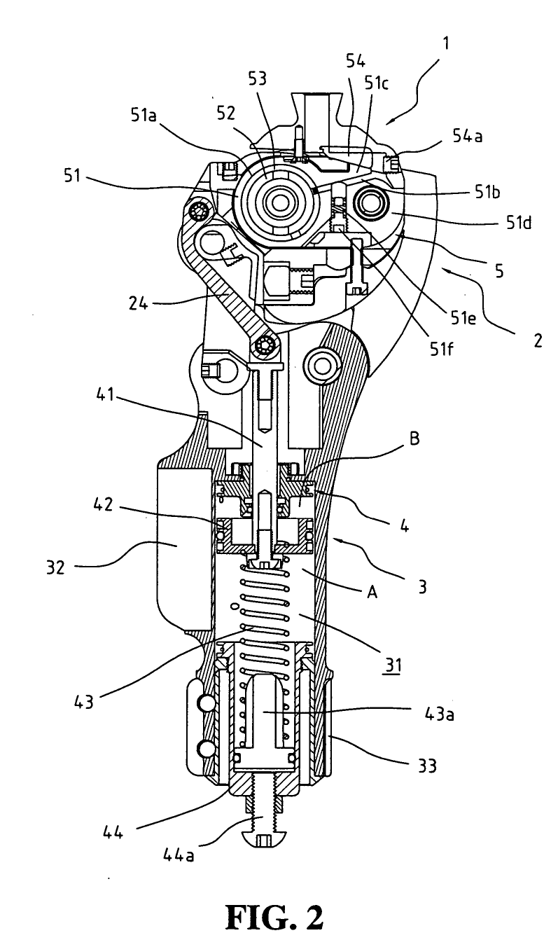

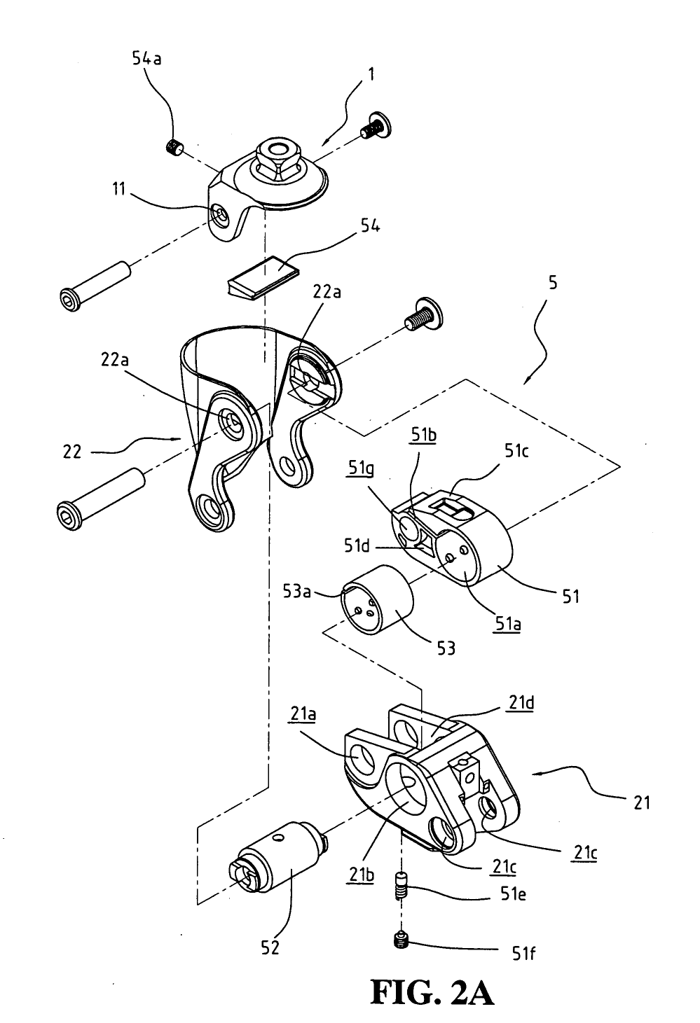

The leg portion 3 has a receiving space 31 defined to receive therein the pneumatic cylinder 4, a side cover 32 to selectively close a window 30 on a side of the leg portion 3 and a connection portion 33 formed on a bottom of the leg portion 3 for connection with a prosthesis. The pneumatic cylinder 4 includes a push rod 41, a piston 42 securely connected to a bottom of the push rod 41, a spring 43 and a first adjusting portion 44 mounted on a bottom opening in the connection portion 33 of the leg portion 3 to close the bottom opening. It is noted especially noted from FIG. 2 that an adjusting block 43a is movably received in the first adjusting portion 44 and the spring 43 is mounted between the piston 42 and the adjusting block 43a. An adjusting screw 44a is threadingly extended into the first adjusting portion 44 and a distal end thereof is abutted against a bottom face of the adjusting block 43a such that rotation of the adjusting screw 44a is able to move the adjusting block 43a inside the receiving space 31. Further, due to the movement of the adjusting block 43a, a recovery force from the spring 43 to the piston 42 is adjusted.

From the above description, it is noted that because the push rod 41 is pivotally connected to the supporting rod 21 and the supporting rod 24 is pivotally connected to the saddle-like body 21 of the knee portion 2 which is connected to the supporting portion 1 so that whenever there is a load on top of the supporting portion 1, the recovery force from the spring 43 is able to balance the load.

With reference to FIG. 2A, it is noted that the saddle-like body 21 of the knee portion 2 has a brake device 5 installed inside the saddle-like body 21. The brake device 5 includes an actuator 51, a core 52, and a wedge 54. The actuator 51 has a first through hole 51a, a slit 51b defined through a periphery of the actuator 51 to communicate with the first through hole 51a and to divide the periphery of the actuator 51 into an upper portion 51c and a lower portion 51d which has a resilient element 51e received therein and a resilient element adjusting screw 51f threadingly connected to a periphery of the lower portion and engaged with an end of the resilient element 51e so that rotation of the resilient element adjusting screw 51f is able to adjust engagement of the other end of the resilient element 51e with the upper portion 51c. The core 52 is rotatably received in the first through hole 51a of the actuator 51 and an outer periphery thereof is in sliding engagement with an inner periphery defining the first through hole 51a of the actuator 51. The wedge 54 is provided between a bottom of the supporting portion 1 and a top face of the upper portion 51c of the actuator 51. A wedge adjusting screw 54a is threadingly extended into a rear face of the supporting portion 1 to abut a face of the wedge 54 such that from the position change of the wedge 54 due to the rotation of the wedge adjusting screw 54a, a load on top of the upper portion 51c from the supporting portion 1 is adjusted.

The saddle-like body 21 has a space 21d defined to receive therein the actuator 51, a second through hole 21a defined in a front portion of the saddle-like body 21 to communicate with a third through hole 51g defined in a front portiori of the actuator 51 and a fourth through hole 11 defined in the supporting portion 1 such that a pin (not numbered) is able to extend through the aligned fourth through hole 11, the second through hole 21a and the third through hole 51g to combine the supporting portion 1, the saddle-like body 21 and the actuator 51. The saddle-like body 21 further has a core hole 21b defined to communicate with the first through hole 51a of the actuator 51 after the actuator 51 is received in the space 21d so that the core 52 is able to be received in the first through hole 51a whose outer periphery is received in the core hole 21b. A rear knee hole 21c is defined in a rear portion of the saddle-like portion 21 to pivotally connect to the rear knee 23. The saddle-like body 21 is securely connected to the front knee 22 via the core 52 received in the upper pivot hole 22a in the front knee 22. Thus the front knee 22 is moved together with the core 52. Therefore, whenever there is a load on top of the supporting portion 1, the load actually on top of the upper portion 51c deforms the upper portion 51c. However, due to the provision of the resilient element 51e and the resilient element adjusting screw 51f to adjust the supporting force to the upper portion 51c from the resilient element 51e due to the rotation of the resilient element adjusting screw 51f, the load on the upper portion 51c is somewhat reduced. Anyhow, the load on top of the upper portion 51c reduces the diameter of the first through hole 51a, which locks the core 52. As a result of the core 52 being locked by the inner periphery of the first through hole 51a, there is no relative pivotal movement between the front knee 22 and the saddle-like body 21.

A friction sleeve 53 is added into the first through hole 51a of the actuator 51 and the core 52 is received in the friction sleeve 53. Therefore, when the load is on top of the upper portion 51c to deform the diameter of the first through hole 51a, the diameter of the friction sleeve 53 is also deformed and thus the core 52 is locked, which causes that the front knee 22 is not able to pivot relative to the saddle-like body 21.

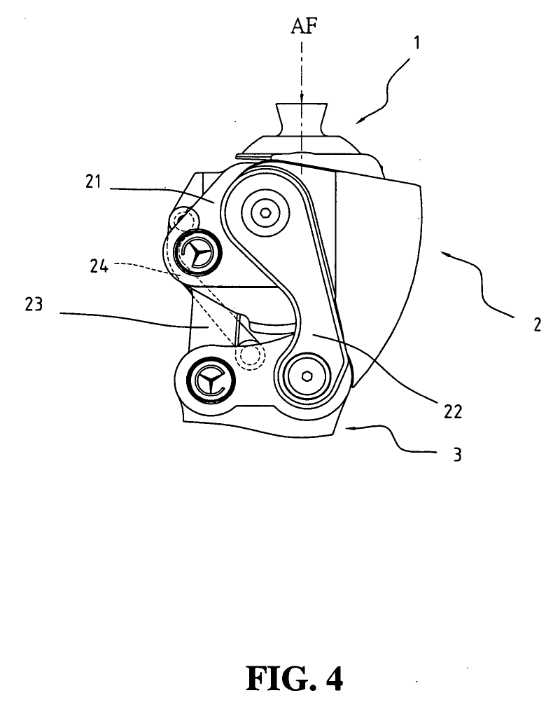

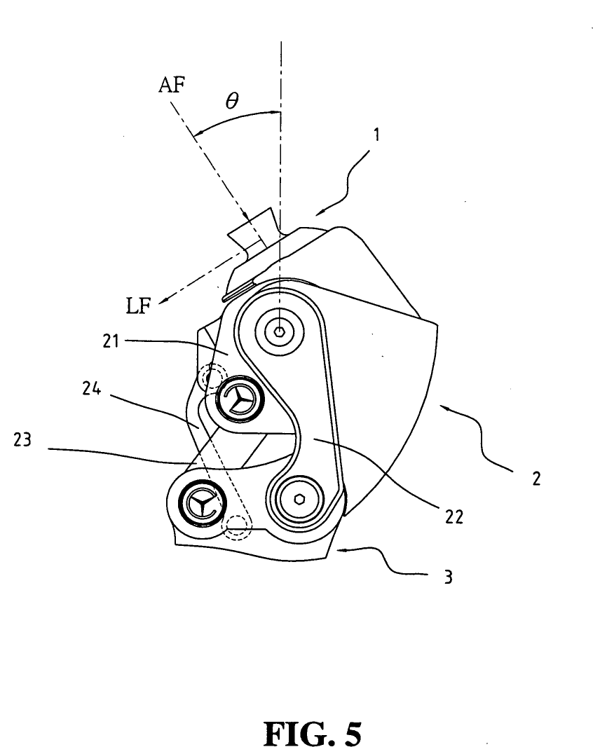

With reference to FIGS. 4 and 5, it is noted that when there is a vertical load on top of the supporting portion 1, the knee portion 2 will not move due to the position of the pivotal connection between the saddle-like body 21 and the front knee 22 being at the rear of a longitudinal axis of the supporting portion 1. Therefore, when there is an axial force (AF) due to the user's body weight on the supporting portion 1, the knee portion 2 is immovable. However, when there is a lateral force (LF) on the supporting portion 1 relative to the longitudinal axis of the supporting portion 1, the knee portion 2 is able to pivot for an angle θ relative to the longitudinal axis of the supporting portion 1. Further, even the axial force continues to apply to the supporting portion 1 to allow the knee portion 2 to pivot, the pivotal connection position between the front knee 22 and the saddle-like body 21 will not exceed the longitudinal axis so as to protect the user's safety. Preferably, the angle θ is about 28 degrees.

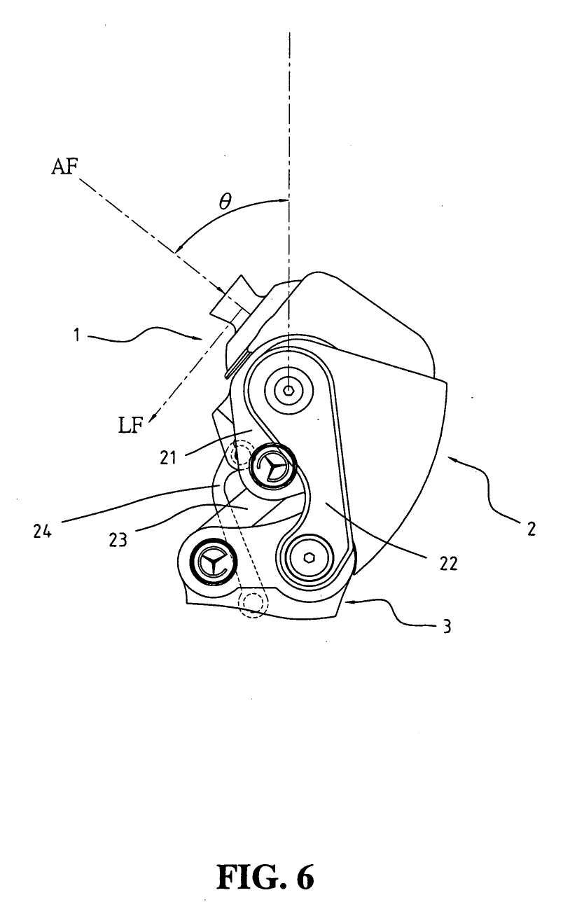

With reference to FIGS. 5 and 6, when the axial force (AF) continues to apply to the supporting portion 1 when the knee portion 2 is pivoted for angle θ, the knee portion 2 will not pivot, as shown in FIG. 5. However, when there is a lateral force (LF) is applying to the supporting portion 1, the knee portion 2 is able to pivot from the position θ to the position Φ, in which the pivotal connection position between the front knee 22 and the saddle-like body 21 is exceeding an extension of the longitudinal axis. Under such a circumstance, either the axial force or the lateral force applied to the supporting portion 1 will cause the knee portion 2 to pivot.

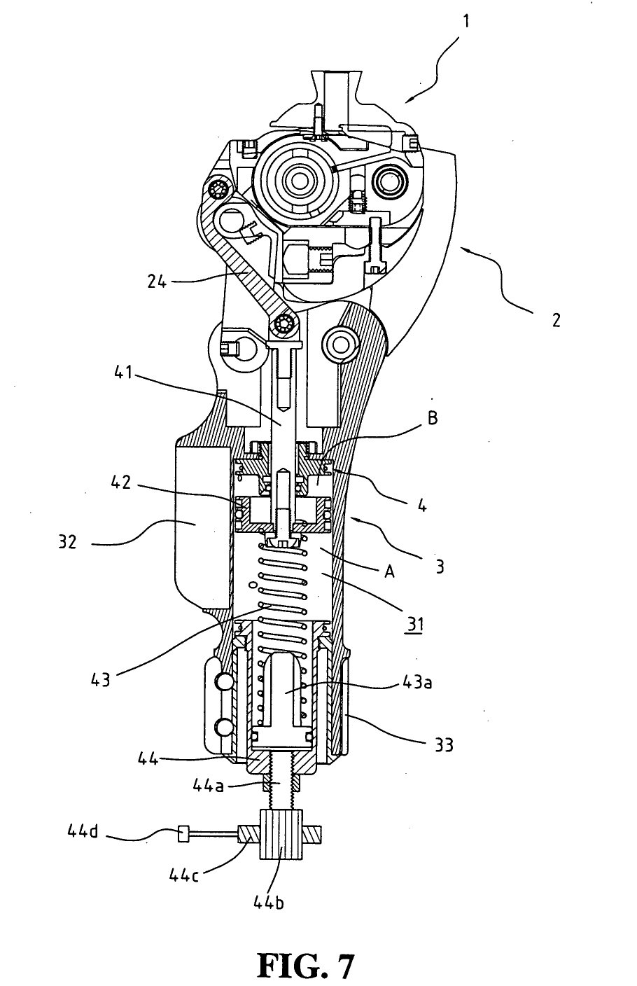

With reference to FIG. 7, a different embodiment of the pneumatic cylinder 4 is shown. The first adjusting portion 44 is used to plug the bottom opening of the receiving space 31 of the leg portion 3. The first adjusting portion 44 has an adjusting screw 44a threadingly extended into the first adjusting portion 44 and having a gear 44b formed on a bottom of the adjusting screw 44a. The gear 44b is mated with a threaded rod 44c which has a knob 44d extended outward therefrom. Therefore, rotation of the knob 44d is able to drive the threaded rod 44c to rotate, which drives the gear 44b to rotate. Accordingly the adjusting screw 44a is moved relative to the first adjusting portion 44. Due to the position change of the adjusting screw 44a, the position of the adjusting block 43a is changed. As a result of the adjusting block 43a position change, the recovery force from the spring 43 is adjusted.

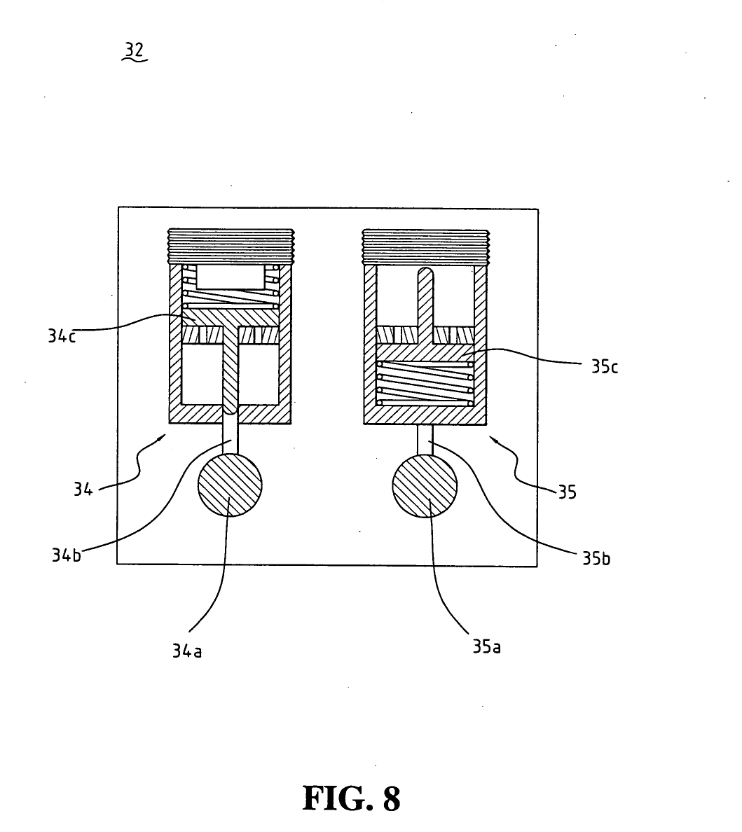

With reference to FIGS. 2 and 8, the side cover 32 is defined to have a second adjusting portion 34 and a third adjusting portion 35. The second adjusting portion 34 has a second adjusting screw 34a threadingly connected to the side cover 32, a second adjusting path 34b and a second adjusting valve 34c. The second adjusting path 34b is selectively communicating with a lower chamber A and an upper chamber B divided by the piston 42 of the pneumatic cylinder 4 inside the receiving space 31 of the leg portion 3. Due to the threading connection of the second adjusting screw 34a to the side cover 32, rotation of the second adjusting screw 34a is able to adjust a cross section area of the second adjusting path 34b allowing air in the lower chamber A to flow into the upper chamber B. Thus when the saddle-like body 21 is pivoted relative to the supporting portion 1 to allow the supporting rod 24 to press the push rod 41 of the pneumatic cylinder 4, downward movement of the piston 42 will force the air in the lower chamber A to flow into the upper chamber B. Because the rotation of the second adjusting screw 34a is able to adjust the cross section area of the second adjusting path 34b, the air flux flowing from the lower chamber A to the upper chamber B is adjusted such that the cushion effect is provided to the saddle-like body 21 of the knee portion 2.

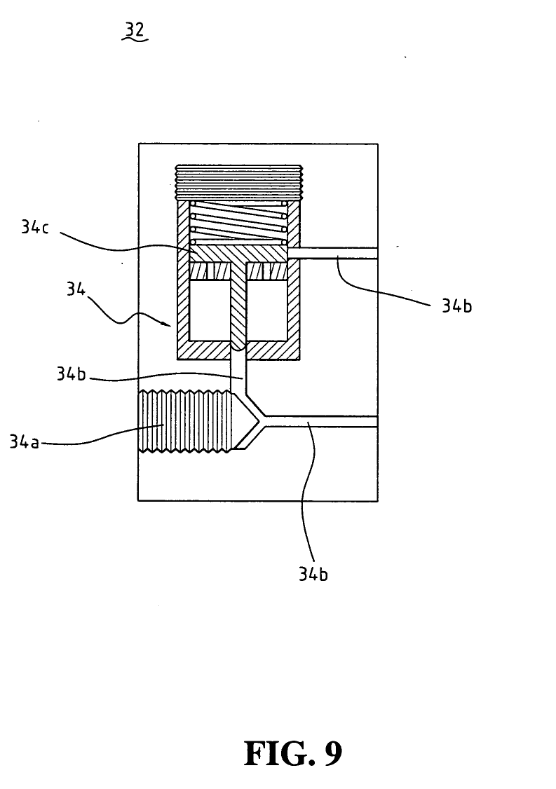

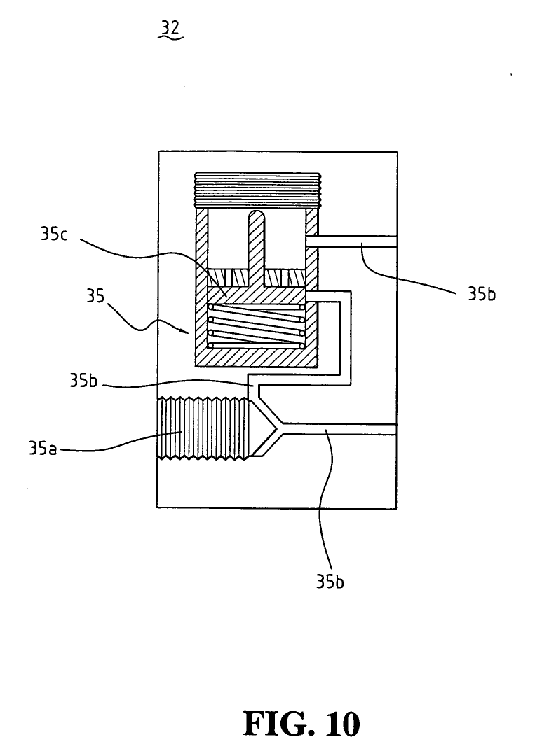

The third adjusting portion 35 has a second adjusting screw 35a threadingly connected to the side cover 32, a second adjusting path 35b and a second adjusting valve 35c. The second adjusting path 35b is selectively communicating with the lower chamber A and the upper chamber B divided by the piston 42 of the pneumatic cylinder 4 inside the receiving space 31 of the leg portion 3. Due to the threading connection of the third adjusting screw 35a to the side cover 32, rotation of the third adjusting screw 35a is able to adjust a cross section area of the third adjusting path 35b allowing air in the upper chamber B to flow into the lower chamber A. Thus when the saddle-like body 21 is pivoted relative to the supporting portion 1 to allow the supporting rod 24 to drive the push rod 41 of the pneumatic cylinder 4 to move upward, upward movement of the piston 42 will force the air in the upper chamber B to flow into the lower chamber A via the third adjusting path 35b. Because the rotation of the second adjusting screw 35a is able to adjust the cross section area of the third adjusting path 35b, the air flux flowing from the upper chamber B to the lower chamber A is adjusted such that the cushion effect is provided to the saddle-like body 21 of the knee portion 2. From the above description, it is noted that the second adjusting portion 34 and the third adjusting portion 35 respectively allows air in the lower (upper) chamber A (B) to flow into the upper (lower) chamber B (A) so as to provide cushion effect to the saddle-like body 21 in either downward or upward movement. With reference to FIGS. 9 and 10 different structural embodiments of the second adjusting portion 34 and the third adjusting portion 35 are shown.

Even though numerous characteristics and advantages of the present invention have been set forth in the foregoing description, together with details of the structure and function of the invention, the disclosure is illustrative only, and changes may be made in detail, especially in matters of shape, size, and arrangement of parts within the principles of the invention to the full extent indicated by the broad general meaning of the terms in which the appended claims are expressed.

What is claimed is:

1. An artificial knee comprising:
a supporting portion adapted for connection to a thigh;
a knee portion having saddle-like body, a front knee pivotally sandwiched between the supporting portion and the saddle-like body and a supporting rod pivotally connected to the supporting portion; and
a brake device received in the knee portion and having an actuator securely connected to the knee portion and having a first through hole defined through the actuator and a slit defined through a periphery of the actuator to communicate with the first through hole and to divide a periphery of the actuator into an upper portion in selective engagement with the supporting portion and a lower portion a core rotatably received in the first through hole such that a load on top of the supporting portion deforms a diameter of the first through hole to lock rotation of the core so as to prevent relative pivotal movement between the saddle-like body and the front knee.

2. The artificial knee as claimed in claim 1 further comprising a wedge adjustably received between the supporting portion and the upper portion of the actuator and a wedge adjusting screw threadingly extended into a rear face of the supporting portion to engage with the wedge so that rotation of the wedge adjusting screw is able to change position of the wedge as to adjust the load on top of the upper portion.

3. The artificial knee as claimed in claim 2 further comprising a resilient element received in the lower portion and a resilient element adjusting screw threadingly connected to a periphery of the lower portion and engaged with an end of the resilient element so that rotation of the resilient element adjusting screw is able to adjust engagement of the other end of the resilient element with the upper portion.

4. The artificial knee as claimed in claim 3 further comprising a friction sleeve received in the first through hole of the actuator to receive therein the core and having a gap corresponding to the slit of the core.

5. An artificial knee comprising:
a supporting portion adapted for connection with a thigh;
a knee portion having a saddle-like body, a front knee and a rear knee, the saddle-like body is pivotally and respectively connected to a respective top of the front knee and the rear knee, a respective bottom of the front knee and the rear knee being pivotally connected to a leg portion so as to form a four-link-rod mechanism imitating the human knee operational track, a supporting rod being pivotally connected to the supporting portion; and
a brake device having an actuator securely connected to and received in the saddle-like body of the knee portion the knee portion and having a first through hole defined through the actuator and a slit defined through a periphery of the actuator to communicate with the first through hole and to divide a periphery of the actuator into an upper portion in selective engagement with the supporting portion and a lower portion a core rotatably received in the first through hole such that a load on top of the supporting portion deforms a diameter of the first through hole to lock rotation of the core so as to prevent relative pivotal movement between the saddle-like body and the front knee.

6. The artificial knee as claimed in claim 5, wherein the leg portion has a receiving space to receive therein a pneumatic cylinder and a connection portion for connection with a limb extension, the pneumatic cylinder having a push rod pivotally connected to the supporting rod and a first adjusting portion mounted to close a bottom opening of the receiving space of the leg portion and having an adjusting block slidably received in the first adjusting portion, a spring received between a piston and the adjusting block and an adjusting screw threadingly extended into the first adjusting portion such that rotation of the adjusting screw is able to move the adjusting block inside the receiving space so as to adjust a recovery force from the spring to the piston of the pneumatic cylinder.

7. The artificial knee as claimed in claim 6 further comprising a wedge adjustably received between the supporting portion and the upper portion of the actuator and a wedge adjusting screw threadingly extended into a rear face of the supporting portion to engage with the wedge so that rotation of the wedge adjusting screw is able to change position of the wedge as to adjust the load on top of the upper portion.

8. The artificial knee as claimed in claim 7 further comprising a resilient element received in the lower portion and a resilient element adjusting screw threadingly connected to a periphery of the lower portion and engaged with an end of the resilient element so that rotation of the resilient element adjusting screw is able to adjust engagement of the other end of the resilient element with the upper portion.

9. The artificial knee as claimed in claim 8 further comprising a friction sleeve received in the first through hole of the actuator to receive therein the core and having a gap corresponding to the slit of the core.

10. The artificial knee as claimed in claim 8, wherein the first adjusting portion has an adjusting screw threadingly extended into the first adjusting portion and having a gear formed on a bottom of the adjusting screw to mate with a threaded rod which has a knob extended outward therefrom such that rotation of the knob is able to drive the threaded rod as well as the gear to rotate, whereby the adjusting screw is moved relative to the first adjusting portion, the position of the adjusting block is also changed such that the recovery force from the spring is adjusted.

11. The artificial knee as claimed in claim 6, wherein the leg portion has a side cover to selectively close a side opening of the leg portion.

12. The artificial knee as claimed in claim 11, wherein the side cover has a second adjusting portion which has a second adjusting screw threadingly connected to the side cover, a second adjusting path and a second adjusting valve, the second adjusting path is selectively communicating with a lower chamber and an upper chamber divided by the piston of the pneumatic cylinder inside the receiving space of the leg portion, so that rotation of the second adjusting screw is able to adjust a cross section area of the second adjusting path allowing air in the lower chamber to flow into the upper chamber and when the saddle-like body is pivoted relative to the supporting portion to allow the supporting rod to press the push rod of the pneumatic cylinder, downward movement of the piston will force the air in the lower chamber to flow into the upper chamber, the air flux flowing from the lower chamber to the upper chamber is adjusted such that the cushion effect provided to the saddle-like body of the knee portion is adjusted.

13. The artificial knee as claimed in claim 12, wherein the front knee is able to pivot relative to the saddle-like body for 28 degrees relative to an axial axis of the supporting portion.

14. The artificial knee as claimed in claim 11, wherein the side cover has a third second adjusting portion which has a third adjusting screw threadingly connected to the side cover, a third adjusting path and a third adjusting valve, the third adjusting path is selectively communicating with a lower chamber and an upper chamber divided by the piston of the pneumatic cylinder inside the receiving space of the leg portion, so that rotation of the third adjusting screw is able to adjust a cross section area of the third adjusting path allowing air in the upper chamber to flow into the lower chamber and when the saddle-like body is pivoted relative to the supporting portion to allow the supporting rod to lift the push rod of the pneumatic cylinder, upward movement of the piston will force the air in the upper chamber to flow into the downward chamber, the air flux flowing from the upper chamber to the lower chamber is adjusted such that the cushion effect provided to the saddle-like body of the knee portion is adjusted.

15. The artificial knee as claimed in claim 12, wherein the side cover has a third second adjusting portion which has a third adjusting screw threadingly connected to the side cover, a third adjusting path and a third adjusting valve, the third adjusting path is selectively communicating with a lower chamber and an upper chamber divided by the piston of the pneumatic cylinder inside the receiving space of the leg portion, so that rotation of the third adjusting screw is able to adjust a cross section area of the third adjusting path allowing air in the upper chamber to flow into the lower chamber and when the saddle-like body is pivoted relative to the supporting portion to allow the supporting rod to lift the push rod of the pneumatic cylinder, upward movement of the piston will force the air in the upper chamber to flow into the downward chamber, the air flux flowing from the upper chamber to the lower chamber is adjusted such that the cushion effect provided to the saddle-like body of the knee portion is adjusted.
